(19) United States
(12) Patent Application Publication
(10) Pub. No.: US 2026/0007462 A1
(43) Pub. Date: Jan. 8, 2026

(54) RF ABLATION DEVICE HAVING RING ELECTRODES FOR CREATING CHANNELS IN TISSUE

(71) Applicant: Boston Scientific Medical Device Limited, Galway (IE)

(72) Inventor: Matthew Paul Joseph DiCicco, Toronto (CA)

(21) Appl. No.: 19/258,640

(22) Filed: Jul. 2, 2025

Related U.S. Application Data
(60) Provisional application No. 63/667,445, filed on Jul. 3, 2024.

(51) Int. Cl. A61B 18/14; A61B 17/00; A61B 18/00; A61B 90/00

(52) U.S. Cl. CPC A61B 18/1492; A61B 2017/00247; A61B 2018/00178; A61B 2018/00577; A61B 2018/00791; A61B 2018/00875; A61B 2090/3966

(57) ABSTRACT

An ablation device includes an elongate body. The elongate body includes a proximal end and an opposite distal end. At least one ablation electrode is located on the elongate body. The at least one ablation electrode is configured to receive energy from a control system and deliver the energy to tissue adjacent the at least one ablation electrode. The at least one ablation electrode configured as a ring surrounding the elongate body. At least one conductor extends from the at least one ablation electrode to the proximal end. The at least one conductor is configured to electrically connect the at least one ablation electrode to the control system. At least one reference is located adjacent the at least one ablation electrode.

### CROSS REFERENCE TO RELATED APPLICATION

The present application claims priority to U.S. Provisional Patent Application No. 63/667,445, filed Jul. 3, 2024, the entire disclosure of which is incorporated herein by reference in its entirety.

### TECHNICAL FIELD

The present disclosure relates to medical systems and methods for ablating tissue in a patient. More specifically, the present disclosure relates to medical systems and methods for ablation of tissue for creating channels, hemodynamic modification, or preventing tissue tearing during procedures.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

The use of RF energy to cut or vaporize biological tissue is a widely used technique in surgical applications. A target tissue can be vaporized using a conductive material that delivers RF energy of a specific voltage, frequency, and current density to it. This can be used to ligate or cut tissue or to access locations in the body or vasculature through non-invasive percutaneous surgery. During percutaneous transseptal access from the right to the left atrium in the heart, for example, it is common to apply RF energy to puncture the interatrial septum (IAS), also referred to as an atrial septum.

In order to perform electrosurgery, generally the cutting electrode is designed to have a smalls surface area, on the order of 1-3 mm², to provide sufficient current density at the desired voltage and frequency to induce the electrosurgical cutting effect, rather than ablation. In some instances of puncturing the IAS, the desired effect is to create a larger hole than is provided by the tip of the RF probe used to deliver the therapy. In these cases, physicians will typically dilate up the hole created by several times its original size. This stretching is to allow for passage/exchange of large-bore diameter sheaths into the left-atrium for end therapy such as ablation or structural heart

therapy. Over stretching of the hole to place large-bore structural heart therapy, including stents and percutaneous pumps, can also result in tearing of the tissue, resulting in hematoma around the access site.

After dilation of the puncture hole in the IAS and completion of the surgery the stretched hole may recover back down to its original size and eventually heal with scar tissue within 2-6 months of the intervention. In some instances, it is advantageous to make the large size of the dilated hole permanent. This can be used to induce a hemodynamic modification such as creation of a permanent Left-heart to Right-heart shunt to off-load pressures in the left atrium and left ventricle as a result of degenerative or functional mitral valve disease.

In some instances, creation of a large hole between chambers of the heart and vasculature may be advantageous. The IAS is noted to be a stretchy and compliant tissue. Other tissues in the heart or body, are likely to tear when dilated if they do not possess these qualities. Over-stretching of tissue for placement of large-bore structural heart therapy, such as placement of a percutaneous pump connecting the left atrium and aorta, for example, could result in tearing of these less compliant tissues. Tearing of the hole could result in hematoma forming at the placement site and result in embolic events or inflammation.

The present invention aims to resolve these issues by simultaneously dilating a target tissue and providing energy to the tissue to cauterize, ablate, or vaporize the tissue. As the surface area of RF electrodes needs to be small in order to induce a vaporization effect it is difficult to immediately create a hole of sufficient size for end-therapy catheter delivery or for creation of a permanent hole. Placement of RF electrode “rings” on the outer surface of a dilator or sheath already intended to dilate the target tissue (i.e. IAS) can be used to permanently vaporize the stretched tissue after initial puncture while keeping the surface area of the electrode low.

### SUMMARY

Example 1 is an ablation device having an elongate body. The elongate body includes a proximal end and an opposite distal end. At least one ablation electrode is located on the elongate body. The at least one ablation electrode is configured to receive energy from a control system and deliver the energy to tissue adjacent the at least one ablation electrode. The at least one ablation electrode configured as a ring surrounding the elongate body. At least one conductor extends from the at least one ablation electrode to the proximal end. The at least one conductor is configured to electrically connect the at least one ablation electrode to the control system. At least one reference is located adjacent the at least one ablation electrode.

Example 2 is the device of Example 1, wherein the at least one reference includes a first reference located proximal of the at least one ablation electrode and a second reference located distal of the at least one ablation electrode.

Example 3 is the device of any of Examples 1 or 2, wherein the at least one reference is a radiopaque marker.

Example 4 is the device of any of Examples 1-3, wherein the at least one reference is a sensor.

Example 5 is the device of Example 4, wherein the sensor is a temperature sensor, impedance sensor, or capacitance sensor.

Example 6 is the device of any of Examples 1-5, wherein the elongate body includes a lumen extending from the proximal end to the distal end, the lumen configured for receiving a medical device or a fluid.

Example 7 is the device of any of Examples 1-6, wherein the elongate body includes a distal portion that reduces in diameter towards the distal end and is configured to dilate tissue.

Example 8 is the device of Example 7, wherein the at least one ablation electrode is located on the distal portion.

Example 9 is the device of Example 8, wherein the at least one ablation electrode includes a plurality of ablation electrodes, each of the plurality of ablation electrodes having a different diameter.

Example 10 is the device of any of Examples 1-9, wherein the elongate body includes a first section having a first constant diameter and a second section having a second constant diameter, the second constant diameter being smaller than the first constant diameter.

Example 11 is the device of Example 10, wherein the elongate body includes a third section having a third constant diameter, the third constant diameter being smaller than the second constant diameter.

Example 12 is the device of Example 11, wherein the at least one ablation electrode includes a first ablation electrode located on the first section, a second ablation electrode located on the second section, and a third ablation electrode located on the third section.

Example 13 is the device of any of Examples 1-12, wherein the elongate body includes a recess for receiving tissue, and the at least one ablation electrode is located in the recess.

Example 14 is the device of Example 13, wherein the elongate body includes a movable portion configured to expose and cover the recess.

Example 15 is the device of Example 14, wherein the at least one ablation electrode is located on a distal face of the movable portion, a bottom of the recess, or the proximal face of an enlarged portion of the elongate body.

Example 16 is an ablation device. The device includes an elongate body having a proximal end and an opposite distal end, and an outer surface extending between the proximal end and the distal end. At least one ablation electrode is located on the elongate body. The at least one ablation electrode is configured to receive energy from a control system and deliver the energy to tissue adjacent the at least one ablation electrode. The at least one ablation electrode configured as a ring surrounding the elongate body. At least one conductor extends from the at least one ablation electrode to the proximal end. The at least one conductor is configured to electrically connect the at least one ablation electrode to the control system. At least one reference is positioned on the outer surface adjacent the at least one ablation electrode.

Example 17 is the device of Example 16, wherein the at least one reference includes a first reference located proximal of the at least one ablation electrode and a second reference located distal of the at least one ablation electrode.

Example 18 is the device of Example 16, wherein the at least one reference is a radiopaque marker.

Example 19 is the device of Example 16, wherein the at least one reference is a sensor.

Example 20 is the device of Example 19, wherein the sensor is a temperature sensor, impedance sensor, or capacitance sensor.

Example 21 is the device of Example 16, wherein the elongate body includes a lumen extending from the proximal end to the distal end, the lumen configured for receiving a medical device or a fluid.

Example 22 is the device of Example 16, wherein the elongate body includes a distal portion that reduces in diameter towards the distal end and is configured to dilate tissue.

Example 23 is the device of Example 22, wherein the at least one ablation electrode is located on the distal portion.

Example 24 is the device of Example 23, wherein the at least one ablation electrode includes a plurality of ablation electrodes, each of the plurality of ablation electrodes having a different diameter.

Example 25 is the device of Example 16, wherein the elongate body includes a first section having a first constant diameter and a second section having a second constant diameter, the second constant diameter being smaller than the first constant diameter.

Example 26 is the device of Example 25, wherein the elongate body includes a third section having a third constant diameter, the third constant diameter being smaller than the second constant diameter.

Example 27 is the device of Example 26, wherein the at least one ablation electrode includes a first ablation electrode located on the first section, a second ablation electrode located on the second section, and a third ablation electrode located on the third section.

Example 28 is the device of Example 16, wherein the elongate body includes a recess for receiving tissue, and the at least one ablation electrode is located in the recess.

Example 29 is the device of Example 28, wherein the elongate body includes a movable portion configured to expose and cover the recess.

Example 30 is the device of Example 29, wherein the at least one ablation electrode is located on a distal face of the movable portion, a bottom of the recess, or the proximal face of an enlarged portion of the elongate body.

Example 31 is an ablation device. The device includes an elongate body having a proximal end and an opposite distal end, an outer surface extending between the proximal end and the distal end, and a distal portion that reduces in diameter towards the distal end. The distal portion that reduces in diameter is configured to dilate tissue. At least one ablation electrode is located on the elongate body. The at least one ablation electrode is configured to receive energy from a control system and deliver the energy to tissue adjacent the at least one ablation electrode. The at least one ablation electrode is configured as a ring surrounding the elongate body. At least one conductor extends from the at least one ablation electrode to the proximal end. The at least one conductor is configured to electrically connect the at least one ablation electrode to the control system. At least one reference is positioned on the outer surface adjacent the at least one ablation electrode.

Example 32 is the device of Example 31, wherein the at least one ablation electrode includes a plurality of ablation electrodes, each of the plurality of ablation electrodes having a different diameter.

Example 33 is the device of Example 31, wherein the elongate body includes a first section having a first constant diameter, a second section having a second constant diameter, and a third section having a third constant diameter, the third constant diameter being smaller than the second constant diameter, and the second constant diameter being smaller than the first constant diameter.

Example 34 is an ablation device. The device includes an elongate body having a proximal end and an opposite distal end, and an annular recess for receiving tissue. At least one ablation electrode is located in the recess. The at least one ablation electrode is configured to receive energy from a control system and deliver the energy to tissue adjacent the at least one ablation electrode. At least one conductor extends from the at least one ablation electrode to the proximal end. The at least one conductor is configured to electrically connect the at least one ablation electrode to the control system.

Example 35 is the device of Example 34, wherein the elongate body includes a movable portion configured to expose and cover the recess, and the at least one ablation electrode is located on a distal face of the movable portion.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the disclosure.

FIG. 2 is perspective view of an ablation device according to an embodiment of the disclosure.

FIG. 3 is a perspective view of an ablation device according to an embodiment of the disclosure.

FIG. 4 is a perspective view of an ablation device according to an embodiment of the disclosure.

FIGS. 5A-5C illustrate an ablation device and method of use according to an embodiment of the disclosure.

FIGS. 6A and 6B illustrate electrode arrangements for use in the embodiment of FIGS. 5A-5C.

FIGS. 7A and 7B illustrate electrodes formed by a plurality of discrete electrodes positioned in a ring formation surrounding the ablation device according to an embodiment of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

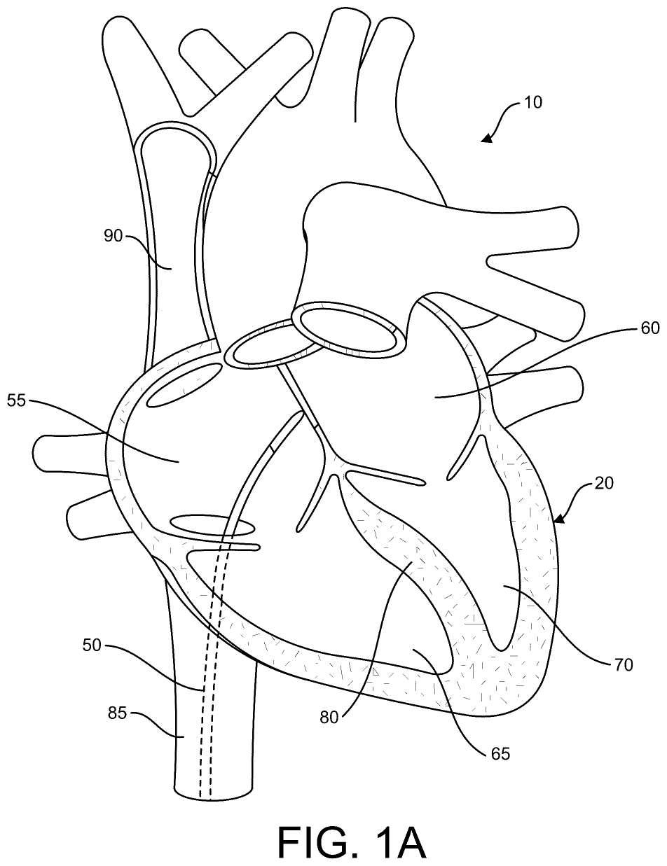

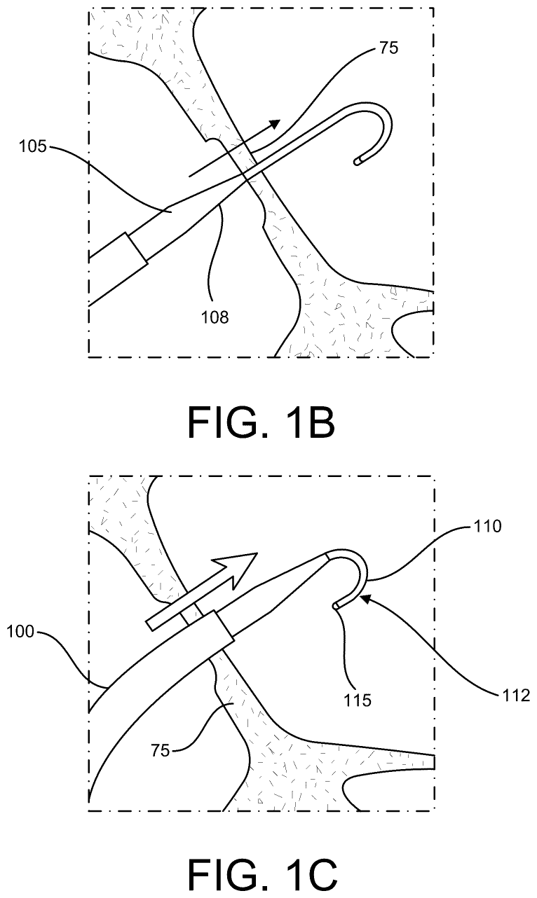

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a radiofrequency (RF) perforation device 110, also known as a piercing device, having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the superior vena cava, the guidewire is removed from the body, and the sheath 100 and the dilator 105 are retracted so that their distal ends are positioned in the right atrium 55. The RF perforation device 110 described can then be introduced into the dilator 105, and advanced toward the heart 20.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the RF generator is electrically coupled to the RF perforation device 110 using a clip that is removable coupled to a proximal portion of the perforation device 110. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 s and about 5 s.

With the tip electrode 115 of the RF perforation device 110 having crossed the atrial septum 75, the dilator 105 can be advanced forward, with the tapered distal tip portion 107 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60 and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

The transseptal access system 50 may be configured to achieve a plurality of different curvatures. This is useful to allow introduction into and positioning of the system 50 at a desired location within the heart 20. For example, the various curvatures allow for achieving desired positioning of the dilator 105 and the RF perforation device 110 along a portion of the atrial septum 75.

In the various embodiments disclosed herein, the perforation device 110 does not include a hub or handle connected to a proximal portion of the wire, which could functional as a positional indicator for the perforation device, for example, by allowing a user to position such a hub or handle with respect to a corresponding structure on the outer member. Thus, to allow a user to properly position an inner member, such as a guidewire or RF perforation device 110, in relationship to the dilator 105, a system of markers may be placed on a proximal portion of the inner member to provide cues to a user. The system of markers may include a plurality of markers positioned symmetrically or asymmetrically along the proximal portion of the inner member. The markers may include a plurality of different colors or different tactile cues The plurality of different colors may include a first color and a second color that are contrasting and easily discernable from one another, for example white and blue.

The markers may provide cues to a user in order to allow for proper positioning of an inner member within an outer member during different steps of a medical procedure. For example, multiple markers can be used to indicate positioning of an inner member such that a distal tip is contained within the outer member, indicate proper positioning of a distal tip during RF delivery, indicate proper positioning of a distal for tenting tissue, indicate a recommended insertion depth into a pulmonary vein, indicate an insertion depth until max rail of the inner member, or indicate when the tip of the inner member is protruding a set distance from the outer member.

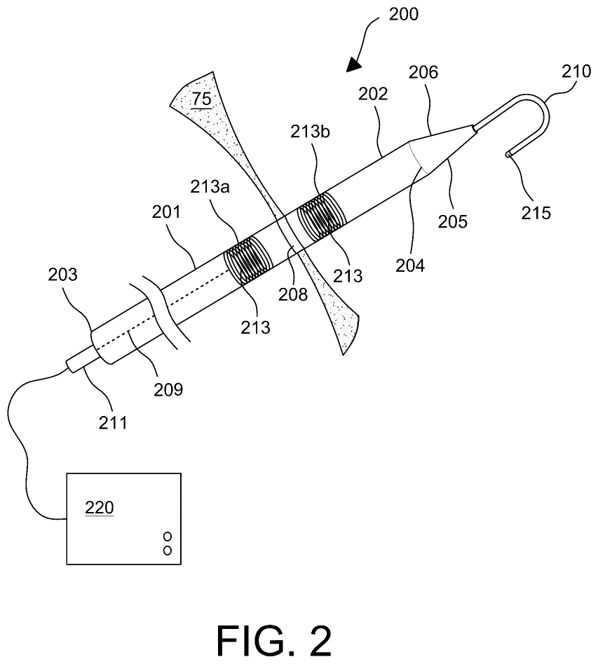

FIG. 2 is perspective view of an ablation device 200 according to an embodiment of the disclosure. The ablation device 200 includes an introducer sheath 201 having an elongate body 202 including a proximal end 203 and an opposite distal end 204. The elongate body 202 includes a lumen through which a dilator 205 having a tapered distal portion 206 configured to dilate tissue is translatable. The lumen can also be used to deliver a fluid, such as a contrast medium, to a location within a body. An RF perforation device 210 is translatable through a lumen of the dilator 205. The RF perforation device 210 includes a distal tip electrode 215 capable of creating a cut or puncture in a body tissue, such as an atrial septum 75. Once the cut or puncture is made, the tapered distal portion 206 can be inserted into the cut or puncture to enlarge the cut or puncture.

The elongate body 202 has an outer surface extending between the proximal end 203 and the distal end 204. At least one ablation electrode 208 is located on the elongate body 202. The at least one ablation electrode 208 is configured to receive energy from a control system 220, such as an RF generator, and deliver the energy to tissue adjacent the at least one ablation electrode 208. In some embodiments, the at least one ablation electrode 208 is configured as a ring and surrounds the elongate body 202. In some embodiments, the at least one ablation electrode 208 is a partial ring and surrounds only a portion of the elongate body 202. For example, a plurality of discrete electrodes, discussed below, can be positioned in a ring formation surrounding the elongate body 202. A benefit to this arrangement allows for an increase in length of each of the plurality of discrete electrodes while keeping the surface area of each of the plurality of discrete electrodes low. Thus, increasing the working area for inducing the RF vaporization effect.

At least one conductor 209 extends from the at least one ablation electrode 208 to the proximal end 203 of the elongate body 202. The at least one conductor 209 is configured to electrically connect the at least one ablation electrode 208 to the control system 220. In the embodiment including a plurality of discrete electrodes, each of the plurality of discrete electrodes are connected to a corresponding conductor. The proximal end 203 of the elongate body 202 may include a connector 211 to removably connect to the control system 220. The at least one conductor 209 can extend through a lumen or channel from the at least one ablation electrode 208 to the connector 211. The at least one conductor 209 can take the form of an insulated or uninsulated wire. If uninsulated, the wire should be located in an insulated channel or lumen to prevent short circuiting. In some embodiments, each ablation electrode 208 can be electrically connected to the control system 220 by a corresponding conductor 209. In some embodiments, a plurality of ablation electrodes 208 may be connected to the control system 220 by a single conductor 209.

In order to help a user position the at least one ablation electrode 208 at a desired tissue location, also known as a target tissue location, at least one reference 213 is positioned adjacent the at least one ablation electrode 208. The at least one reference 213 can be placed on the outer surface of the elongate body 202 in proximity to the at least one ablation electrode 208. The least one reference 213 can include a first reference 213a located proximal of the at least one ablation electrode 208 and a second reference 213b located distal of the at least one ablation electrode 208.

The at least one reference 213 is configured to provide information to a user that allows the user to place the at least one ablation electrode 208 at a desired location. In some embodiments, the at least one reference 213 is a marker, such as a radiopaque marker, that can be viewed by an imaging modality. When viewed under imaging, a user would manipulate the elongate body 202 to place the first reference 213a and the second reference 213b on opposite sides of a target tissue, thus placing the at least one ablation electrode 208 adjacent the target tissue.

In some embodiments, the at least one reference 213 is a sensor. The sensor can provide information to a user to aid in positioning the at least one ablation electrode 208. In some aspects, the sensor can provide location information, such as gps, or other location information relative to a known coordinate system or datum. In some aspects, the sensor can provide physiological information to a user to help identify a target tissue. For example, the sensor can include a temperature sensor that can determine temperature differences between various tissue structures. In some embodiments, the sensor can include impedance sensor or capacitance sensor that can identify different aspects of a target tissue, such as a blood pool adjacent a target tissue.

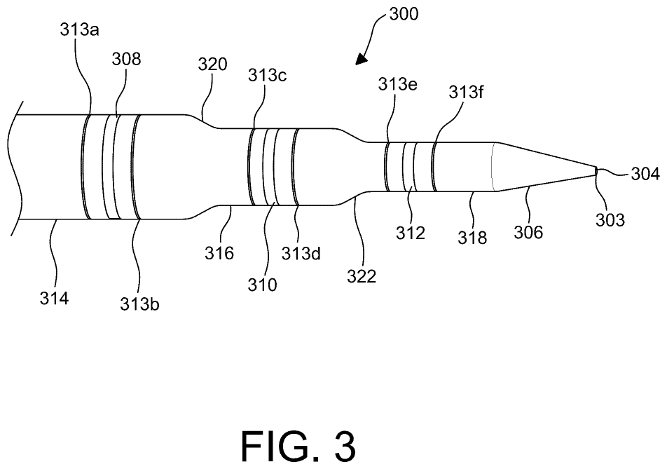

FIG. 3 is a perspective view of an ablation device 300 according to an embodiment of the disclosure. The ablation device 300 is configured as a dilator and includes an elongate body 302 having various diameters and a tapered distal portion 306. The tapered distal portion 306 reduces in diameter towards a distal end 304 and is configured to dilate tissue. The ablation device 300 includes a lumen 303 through which a medical device, such as a perforation device, is translatable. Additionally, the lumen can also be used to deliver a fluid, such as a contrast medium, to a location within a body.

The elongate body 302 includes a first section 314 having a first constant diameter, a second section 316 having a second constant diameter, and a third section 318 having a third constant diameter. The second constant diameter is smaller than the first constant diameter, and the third constant diameter is smaller than the second constant diameter. In one embodiment, the first section 314, the second section 316, and the third section 318 each have the same length. In another embodiment, the first section 314, the second section 316, and the third section 318 each have different lengths. The first section 314 extends from a first transition 320 to a proximal end of the elongate body 302. The second section 316 extends from a second transition 322 to the first transition 320. The third section 318 extends from the tapered distal portion 306 to the second transition 322.

The ablation device 300 includes a plurality of ablation electrodes 308, 310, 312. The plurality of ablation electrodes 308, 310, 312 are electrically connected to a control system by at least one conductor (not shown) passing through the elongate body 302 to a proximal connector (not shown). The plurality of ablation electrodes 308, 310, 312 are configured as ring electrodes that circumscribe the elongate body 302. In some embodiments, the electrodes 308, 310, 312 are positioned in recesses in order to ensure a smooth outer surface along the length of the device 300. In some embodiments, the electrodes 308, 310, 312 are formed of thin bands of an electrically conductive material, or of a polymeric material doped with electrically conductive particles.

Each of the electrodes 308, 310, 312 have different diameters. A first ablation electrode 308 is located on the first section 314. The first ablation electrode 308 has a diameter that is substantially identical to the diameter of the first section 314. A second ablation electrode 310 is located on the second section 316. The second ablation electrode 310 has a diameter that is substantially identical to the diameter of the second section 316. A third ablation electrode 312 is located on the third section 318. The third ablation electrode 312 has a diameter that is substantially identical to the diameter of the third section 318.

The device 300 includes at least one reference for providing information to a user that allows the user to place any of the ablation electrodes 308, 310, 312 at a desired tissue location. As discussed above the at least one reference can include radiopaque markers or sensors. As illustrated in FIG. 3, the at least one reference includes a plurality of references 313a, 313b, 313c, 313d, 313e, 313f associated with each of the ablation electrodes 308, 310, 312. The first ablation electrode 308 includes a proximal reference 313a and a distal reference 313b. The second ablation electrode 310 includes a proximal reference 313c and a distal reference 313d. The third ablation electrode 312 includes a proximal reference 313e and a distal reference 313f.

The device 300 can be used to sequentially dilate and vaporize a target tissue. This allows for a stepwise creation of a hole or opening through a target tissue. This is advantageous when tissue is non-compliant or where there is concern of the target tissue tearing during advancement of devices through the hole or opening. During use, a perforation device is used to make an initial puncture through a target tissue. The tapered distal portion 306 is inserted into the puncture and used to dilate the tissue. Using the third ablation electrode proximal reference 313e and/or distal reference 313f, a user aligns the third ablation electrode 312 with the tissue and energy is applied to the electrode 312 to cauterize and/or vaporize the tissue adjacent the electrode 312. Next, the device 300 is further advanced into the opening to stretch the tissue along the second transition 322 to the second section 316. Using the second ablation electrode proximal reference 313c and/or distal reference 313d, a user aligns the second ablation electrode 310 with the tissue and energy is applied to the electrode 310 to cauterize and/or vaporize the tissue adjacent the second ablation electrode 310. Next, the device 300 is further advanced into the opening to stretch the tissue along the first transition 320 to the first section 314. Using the first ablation electrode proximal reference 313a and/or distal reference 313b, a user aligns the first ablation electrode 308 with the tissue and energy is applied to the electrode 308 to cauterize and/or vaporize the tissue adjacent the first ablation electrode 308.

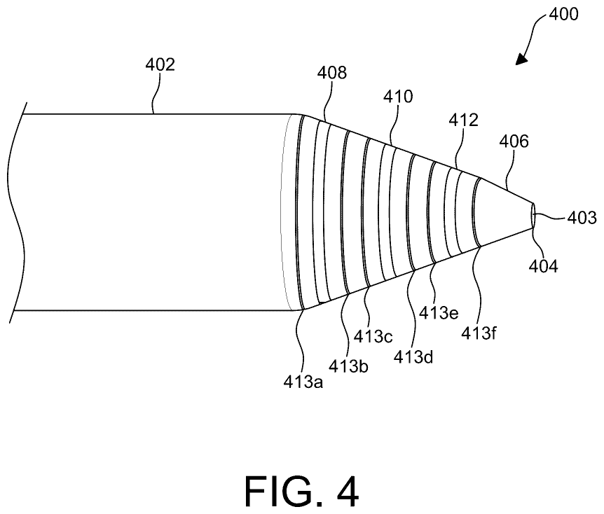

FIG. 4 is a perspective view of an ablation device 400 that also allows for stepwise creation of a hole or opening through a target tissue, according to an embodiment of the disclosure. The ablation device 400 is configured as a dilator and includes an elongate body 402 having a tapered distal portion 406. The tapered distal portion 406 reduces in diameter towards a distal end 404 and is configured to dilate tissue. At least one ablation electrode is located on the distal portion 406. As illustrated in FIG. 4, the at least one ablation electrode includes a plurality of ablation electrodes 408, 410, 412. The plurality of ablation electrodes 408, 410, 412 are arranged along the tapered distal portion 496 such that each of the plurality of ablation electrodes 408, 410, 412 has a different diameter. The plurality of ablation electrodes includes a first ablation electrode 408 having a first diameter, a second ablation electrode 410 having a second diameter, and a third ablation electrode 412 having a third diameter. The first diameter is larger than the second, and the second is larger than the third.

The ablation device 400 includes a lumen 403 through which a medical device, such as a perforation device, is translatable. Additionally, the lumen 403 can also be used to deliver a fluid, such as a contrast medium, to a location within a body.

The plurality of ablation electrodes 408, 410, 412 are electrically connected to a control system by at least one conductor (not shown) passing through the elongate body 402 to a proximal connector (not shown). The plurality of ablation electrodes 408, 410, 412 are configured as ring electrodes that circumscribe the tapered distal portion 406. In some embodiments, the electrodes 408, 410, 412 are positioned in recesses in order to ensure a smooth outer surface along the length of the tapered distal portion 406. In some embodiments, the electrodes 408, 410, 412 are formed of thin bands of an electrically conductive material, or of a polymeric material doped with electrically conductive particles. In some embodiments, the electrodes 408, 410, 412 partially circumscribe the tapered distal portion 406.

The device 400 includes at least one reference for providing information to a user that allows the user to place any of the ablation electrodes 408, 410, 412 at a desired tissue location. As discussed above the at least one reference can include radiopaque markers or sensors. As illustrated in FIG. 4, the at least one reference includes a plurality of references 413a, 413b, 413c, 413d, 413e, 413f associated with each of the ablation electrodes 408, 410, 412. While a plurality of references are illustrated, it is understood that because of the close proximity of the electrodes 408, 410, 412, a single reference could be used to initially align the third electrode 412 with a target tissue.

The first ablation electrode 408 includes a proximal reference 413a and a distal reference 413b. The second ablation electrode 410 includes a proximal reference 413c and a distal reference 413d. The third ablation electrode 412 includes a proximal reference 413e and a distal reference 413f.

The device 400 can be used to sequentially dilate and vaporize a target tissue. This allows for a stepwise creation of a hole or opening through a target tissue. This is advantageous when tissue is non-compliant or where there is concern of the target tissue tearing during advancement of devices through the hole or opening. Following the creating of an initial puncture through a target tissue, the tapered distal portion 406 is inserted into the puncture and used to dilate the tissue. Using the third ablation electrode proximal reference 413e and/or distal reference 413f, a user aligns the third ablation electrode 412 with the tissue and energy is applied to the electrode 412 to cauterize and/or vaporize the tissue adjacent the electrode 412. Next, using the second ablation electrode proximal reference 413c and/or distal reference 413d, a user aligns the second ablation electrode 410 with the tissue and energy is applied to the electrode 410 to cauterize and/or vaporize the tissue adjacent the second ablation electrode 410. While further advancing the device 400 distally, a user aligns the first ablation electrode 408 with the tissue and energy is applied to the electrode 408 to cauterize and/or vaporize the tissue adjacent the first ablation electrode 408.

In some embodiments, the ablation electrodes of FIGS. 2, 3, and 4 could be configured as capacitance/impedance sensors. In such an arrangement, when tissue contact is detected by one of the ablation electrodes, energy can be applied to the ablation electrode to cauterize and/or vaporize the tissue adjacent to the ablation electrode. This would allow a user to advance through the target tissue while the system automatically vaporizes the tissue. For example, as the device is advanced through a target tissue, such as an atrial septum, the capacitance/impedance sensors may relay information to the control system 220. The control system 220 analyses the information to determine when a desired ablation electrode is positioned adjacent the target tissue. When a desired ablation electrode is so positioned, the control system 220 delivers energy to the electrode to ablate/cauterize/vaporize the tissue as desired. Upon further movement through the target tissue, the control system 20 determines that the ablation electrode is no longer adjacent the target tissue and automatically stops delivering energy to the electrode.

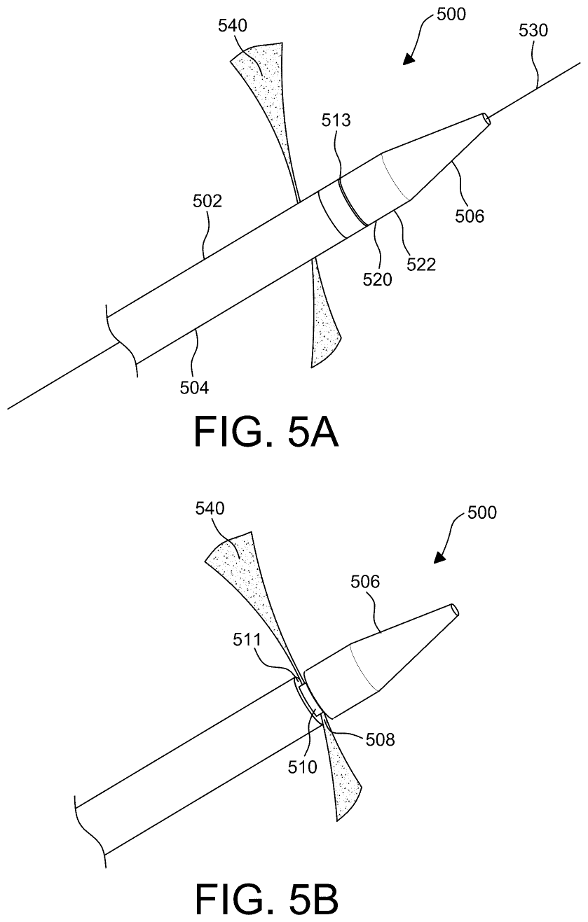

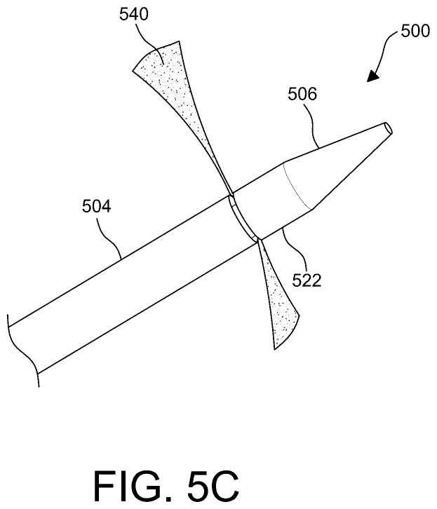

FIGS. 5A-5C illustrate an ablation device 500 and method of use according to an embodiment of the disclosure. The ablation device 500 includes an elongate body 502 having a movable portion 504 and a tapered distal portion 506. The movable portion 504 is translatable along a longitudinal axis 530 of the device 500 in order to expose or cover an annular recess 508. The recess 508 is configured to receive a target tissue when exposed. As such, a target tissue enters the recess 508 when the movable portion 504 is translated to expose the recess.

In some embodiments, the movable portion 504 is a sheath that is translatable along an inner member 520. The inner member 520 includes an enlarged portion 522 having a diameter that is substantially identical to the diameter of the movable portion 504. The enlarged portion 522 is located proximal of the tapered distal portion 506. A recess 508 is formed as the movable portion 504 is moved relative to the enlarged portion 522.

At least one ablation electrode 510 is located in the recess 508. As such, the ablation electrode 510 can deliver energy to tissue located in the recess 508 in order to cauterize or vaporize the tissue. The at least one ablation electrode 510 can be positioned on a portion of the movable portion 504 or the inner member 520. In some embodiments, the at least one ablation electrode 510 is located on the bottom of the recess 508. The electrode 510 takes the form of a ring electrode and circumscribes at least a portion of the inner member 520. In other embodiments, the at least one ablation electrode 510 is positioned on a distal face 511 of the movable portion 504. In this arrangement, the electrode 510 is placed against tissue within the recess 508 by translating the movable portion 504 towards the enlarged portion 522 to pinch the tissue. In other embodiments, the at least one ablation electrode 510 is formed on a proximal face of the enlarged portion 522.

At least one reference 513 is located on the elongate body 502 to aid a user in placing the recess 508 at a desired tissue location. FIG. 5A illustrates the device 500 after having been inserted into a target tissue 540 such as an atrial septum, artery, or other tissue into which a channel is desired to be formed. In FIG. 5A, the movable portion 504 is pressed against the enlarged portion 522 and covers the recess 522. The recess is located distal of the target tissue 540. Using the at least one reference 513, a user positions the recess 508 adjacent the target tissue 540 and translates the movable portion 504 to expose the recess 508 as illustrated in FIG. 5B. One tissue has entered the recess 508, the movable portion 504 is translated towards the enlarged portion 522 to pinch or hold the target tissue 540 as illustrated in FIG. 5C. Energy is delivered to the ablation electrode 510 located in the recess or on the distal face 511 of the movable portion 504 to cauterize or vaporize tissue adjacent the ablation electrode 510.

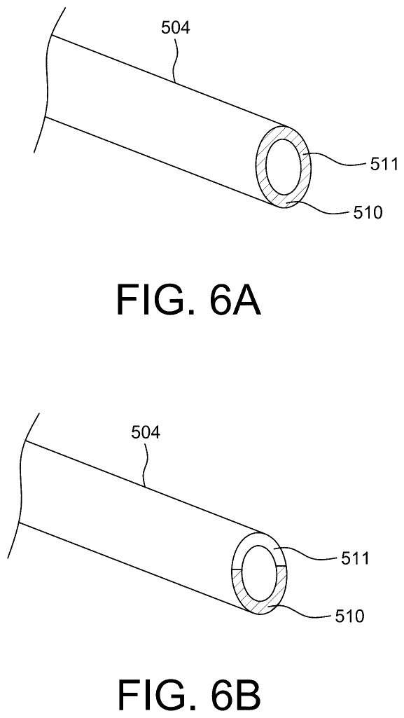

FIGS. 6A and 6B illustrate electrode arrangements for use on a distal face 511 of a movable portion 504 in the embodiment of FIGS. 5A-5C. FIG. 6A illustrates an electrode 510 forming a ring around the entire surface of the distal face 511. This would allow for complete cauterization or vaporization of tissue located in the recess 508. The electrode 510 illustrated in FIG. 6A is located on a portion of the distal face 511. The electrode 510 forms a semi-circular or half-moon shape. This allows for the creation of a flap of material in the target tissue by only cauterizing or vaporizing a portion of the tissue.

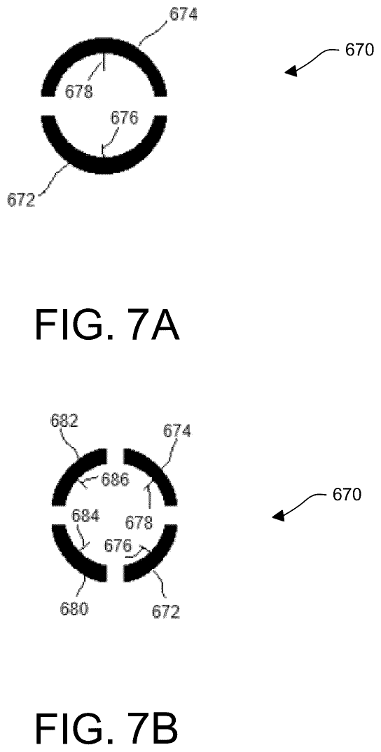

In some embodiments, the ablation electrodes of FIGS. 2, 3, and 4 are formed as segmented electrodes. FIGS. 7A and 7B illustrate segmented electrodes formed by a plurality of discrete electrodes positioned in a ring formation surrounding the ablation device according to an embodiment of the disclosure. A segmented electrode 670 comprises a plurality of discrete electrodes that share a common position longitudinally along the elongate body. This allows for an increase in length of each of the plurality of discrete electrodes while keeping the surface area of each of the plurality of discrete electrodes low. A segmented electrode 670 can take the form of a ring electrode that has been separated into two or more segments, each segment being connected to an individual conductor to allow for separate operation of each segment. While FIGS. 7A and 7B illustrate the at least one segmented electrode as having a circular cross-section, in some embodiments the at least one segmented electrode includes an oval, square, rectangular, or polygonal cross-section.

As illustrated in FIG. 7A, the at least one segmented electrode 670 comprises a first electrode 672 and second electrode 674, the first and second electrode are positioned at the same longitudinal position along the ablation device. A first conductor 676 is mechanically and electrically connected to the first electrode 672 of the segmented electrode 670 and a second conductor 678 is mechanically and electrically connected to the second electrode 674 of the segmented electrode 670. The first conductor 676 and the second conductor 678 electrically couple the first electrode 672 and the second electrode 674, respectively, to the control system 220. In operation, the first and second electrode can operate together or independently. The first electrode 672 and the second electrode 674 can be operated independently, to receive signals from different locations of tissue, or as a pair, for example a bipolar pair during the delivery of energy.

As illustrated in FIG. 7B, the at least one segmented electrode 670 comprises a first electrode 674, a second electrode 672, a third electrode 680, and a fourth electrode 682 positioned at the same longitudinal position along a portion of the ablation device. A first conductor 672 is mechanically and electrically connected to the first electrode 674 of the segmented electrode 670, a second conductor 678 is mechanically and electrically connected to the second electrode 674 of the segmented electrode 670, a third conductor 684 is mechanically and electrically connected to the third electrode 680 of the segmented electrode 670, and a fourth conductor 686 is mechanically and electrically connected to the fourth electrode 682 of the segmented electrode 670. The conductors electrically couple the electrodes to the control system 220. In operation, the first 672, second 674, third 680, and fourth 682 electrode can operate together or independently. Any two of the first 672, second 674, third 680, and fourth 682 electrodes can be operated as a first pair, for example a bipolar pair during the delivery of energy, and the second two can be operated as a second pair.

While FIG. 7A illustrates a segmented electrode 670 as having two independent electrodes and FIG. 7B illustrates a segmented electrode 670 having four independent electrodes, the at least one segmented electrode 670 may include any number of electrodes greater than two. In some embodiments, the at least one segmented electrode 670 may include up to 10 electrodes. In some embodiments, the at least one segmented electrode 670 may include up to 20 electrodes. In some embodiments, the plurality of the electrodes forming the at least one segmented electrode 670 can each be wired independently to allow for independent operation of each of the plurality of electrodes. In some embodiments, a first set of the plurality of electrodes forming the at least one segmented electrode 670 can be wired to a first common conductor to allow for simultaneous operation of the first set, and a second set of the plurality of electrodes forming the at least one segmented electrode can be wired to a second common conductor to allow for simultaneous operation of the second set.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. An ablation device, the device comprising:
an elongate body having a proximal end and an opposite distal end, an outer surface extending between the proximal end and the distal end;
at least one ablation electrode located on the elongate body, the at least one ablation electrode configured to receive energy from a control system and deliver the energy to tissue adjacent the at least one ablation electrode, the at least one ablation electrode configured as a ring surrounding the elongate body;
at least one conductor extending from the at least one ablation electrode to the proximal end, the at least one conductor configured to electrically connect the at least one ablation electrode to the control system; and
at least one reference positioned on the outer surface adjacent the at least one ablation electrode.

2. The device of claim 1, wherein the at least one reference includes a first reference located proximal of the at least one ablation electrode and a second reference located distal of the at least one ablation electrode.

3. The device of claim 1, wherein the at least one reference is a radiopaque marker.

4. The device of claim 1, wherein the at least one reference is a sensor.

5. The device of claim 4, wherein the sensor is a temperature sensor, impedance sensor, or capacitance sensor.

6. The device of claim 1, wherein the elongate body includes a lumen extending from the proximal end to the distal end, the lumen configured for receiving a medical device or a fluid.

7. The device of claim 1, wherein the elongate body includes a distal portion that reduces in diameter towards the distal end and is configured to dilate tissue.

8. The device of claim 7, wherein the at least one ablation electrode is located on the distal portion.

9. The device of claim 8, wherein the at least one ablation electrode includes a plurality of ablation electrodes, each of the plurality of ablation electrodes having a different diameter.

10. The device of claim 1, wherein the elongate body includes a first section having a first constant diameter and a second section having a second constant diameter, the second constant diameter being smaller than the first constant diameter.

11. The device of claim 10, wherein the elongate body includes a third section having a third constant diameter, the third constant diameter being smaller than the second constant diameter.

12. The device of claim 11, wherein the at least one ablation electrode includes a first ablation electrode located on the first section, a second ablation electrode located on the second section, and a third ablation electrode located on the third section.

13. The device of claim 1, wherein the elongate body includes a recess for receiving tissue, and the at least one ablation electrode is located in the recess.

14. The device of claim 13, wherein the elongate body includes a movable portion configured to expose and cover the recess.

15. The device of claim 14, wherein the at least one ablation electrode is located on a distal face of the movable portion, a bottom of the recess, or the proximal face of an enlarged portion of the elongate body.

16. An ablation device, the device comprising:
an elongate body having a proximal end and an opposite distal end, an outer surface extending between the proximal end and the distal end, and a distal portion that reduces in diameter towards the distal end and is configured to dilate tissue;
at least one ablation electrode located on the elongate body, the at least one ablation electrode configured to receive energy from a control system and deliver the energy to tissue adjacent the at least one ablation electrode, the at least one ablation electrode configured as a ring surrounding the elongate body;
at least one conductor extending from the at least one ablation electrode to the proximal end, the at least one conductor configured to electrically connect the at least one ablation electrode to the control system; and
at least one reference positioned on the outer surface adjacent the at least one ablation electrode.

17. The device of claim 16, wherein the at least one ablation electrode includes a plurality of ablation electrodes, each of the plurality of ablation electrodes having a different diameter.

18. The device of claim 16, wherein the elongate body includes a first section having a first constant diameter, a second section having a second constant diameter, and a third section having a third constant diameter, the third constant diameter being smaller than the second constant diameter, and the second constant diameter being smaller than the first constant diameter.

19. An ablation device, the device comprising:
an elongate body having a proximal end and an opposite distal end, and an annular recess for receiving tissue;
at least one ablation electrode located in the recess, the at least one ablation electrode configured to receive energy from a control system and deliver the energy to tissue adjacent the at least one ablation electrode; and
at least one conductor extending from the at least one ablation electrode to the proximal end, the at least one conductor configured to electrically connect the at least one ablation electrode to the control system.

20. The device of claim 19, wherein the elongate body includes a movable portion configured to expose and cover the recess, and the at least one ablation electrode is located on a distal face of the movable portion.
